(19) United States
(12) Patent Application Publication
(10) Pub. No.: US 2026/0225089 A1
(43) Pub. Date: Aug. 6, 2026

(54) A CATALYST COMPOSITION FOR IMPROVING TIME TO REACH HIGH ESTERIFICATION CONVERSION OF CARBOXYLIC ACIDS/ANHYDRIDES

(71) Applicants: Wisconsin Alumni Research Foundation, Madison, WI (US); PPG INDUSTRIES OHIO, INC., Cleveland, OH (US)

(72) Inventors: Ive HERMANS, Middleton, WI (US); Jacob JANSEN, Madison, WI (US); Adam POWELL, Pittsburgh, PA (US); Sarah SPECHT, Pittsburgh, PA (US)

(21) Appl. No.: 19/142,486

(22) PCT Filed: Dec. 21, 2023
(86) PCT No.: PCT/US2023/085382

§ 371 Date: Jun. 23, 2025

Related U.S. Application Data
(60) Provisional application No. 63/434,383, filed on Dec. 21, 2022.

(51) Int. Cl. B01J 31/22; B01J 37/04

(52) U.S. Cl. CPC B01J 31/2226; B01J 37/04; B01J 2231/49; B01J 2531/46; B01J 2531/48; B01J 2531/54

(57) ABSTRACT

Disclosed herein are catalytic systems and methods of using the same for preparing a compound comprising an ester group. The catalytic system comprises a first catalyst comprising a metal selected from bismuth, aluminum, titanium, zirconium, and combinations thereof, and a second catalyst comprising titanium, zirconium, and combinations thereof. The method of using the catalytic system for preparing a compound comprising an ester group comprises reacting one or more ester-forming reactants in the presence of the catalytic system under conditions sufficient to prepare the compound comprising the ester group, wherein the one or more ester-forming reactants comprise a compound having a carboxyl group, a hydroxyl group, an anhydride group, or combinations thereof.

### CROSS-REFERENCE TO RELATED APPLICATIONS

This application claims benefit of priority to U.S. Provisional Application 63/434,383, filed Dec. 21, 2022, the contents of which are incorporated herein in its entirety.

### BACKGROUND

Conventional industrial esterification reactions leverage tin or antimony-containing catalysts. While efficient, both tin and antimony can be toxic. Alternative catalytic systems that have been developed to address the toxicity issues present in the traditional tin or antimony-containing catalysts include titanates/zirconates (i.e., TYZORS). However, such catalytic systems are known for being unstable, such as in water, which is one of the byproducts of esterification reactions. As a result, there exists a need for new catalytic systems that are effective, non-toxic, and exhibit stability in the presence of water.

### SUMMARY OF THE INVENTION

Disclosed herein are catalytic systems and methods of using the same for preparing a compound comprising an ester group. One aspect of the invention provides for a catalytic system. The catalytic system comprises a first catalyst comprising bismuth, aluminum, titanium, zirconium, or combinations thereof, and a second catalyst comprising titanium, zirconium, or combinations thereof.

Another aspect of the invention provides for a method of preparing the catalytic system as described herein. The method comprises mixing the first catalyst and the second catalyst.

Another aspect of the invention provides for preparing a compound comprising an ester group. The method comprises reacting one or more ester-forming reactants in the presence of the catalytic system as described herein under conditions sufficient to prepare the compound comprising the ester group, wherein the one or more ester-forming reactants comprise a compound having a carboxyl group, a hydroxyl group, an anhydride group, or combinations thereof.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1A demonstrates that Ti/Bi catalytic systems in hexane show improved stability in water. 10 equivalents of water per equivalent of the catalytic system are used in the figure. Catalytic system in the vials from left to right: 100% bismuth neodecanoate; 1:4 titanium butoxide to bismuth neodecanoate; 1:2 titanium butoxide to bismuth neodecanoate; 1:1 titanium butoxide to bismuth neodecanoate; 2:1 titanium butoxide to bismuth neodecanoate; 4:1 titanium butoxide to bismuth neodecanoate; 100% titanium butoxide.

FIG. 1B demonstrates the stability test of titanium butoxide/bismuth neodecanoate having a molar ratio of 1:1 in from 10 to up to 1000 equivalents of water.

FIG. 2A shows the cloud-point of the catalytic systems in THE with water. Catalytic systems in the bar graph from left to right: 100% bismuth neodecanoate; 1:4 titanium butoxide to bismuth neodecanoate; 1:2 titanium butoxide to bismuth neodecanoate; 1:1 titanium butoxide to bismuth neodecanoate; 2:1 titanium butoxide to bismuth neodecanoate; 4:1 titanium butoxide to bismuth neodecanoate; and 100% titanium butoxide.

FIG. 2B shows the cloud-point of the various catalytic systems in THE with water tested at 1-10, 20, 30, 40, and 100 equivalents of water.

FIG. 3A shows an exemplary esterification reaction employing the catalytic system of the present invention.

FIG. 3B shows that catalytic systems comprising Ti and Bi have high half-ester conversion.

FIG. 4 shows a plot of the percent conversion to the ester product at 4-hour time point using different catalytic systems containing varied mole fractions of titanium.

FIG. 5 shows a plot of the percent conversion to the ester product at 4-hour time point using varied catalytic systems.

FIG. 6 shows a plot of the percent conversion to the ester product at 4-hour time point compared to how much water is added to the catalytic system.

### DETAILED DESCRIPTION OF THE INVENTION

Disclosed herein are catalytic systems for esterification reactions. The present technology provides for catalytic systems that exhibit improved stability towards deactivation by water while maintaining catalytic activity even when low amounts of metals are included in combination with one another. The catalyst systems described herein have the advantage of reducing the reaction time to reach high esterification conversion while not containing either organic or inorganic tin. Although tin catalysts are known to be quite effective and stable in the presence of water generated during esterification reactions, they are coming under increasing scrutiny for toxicological and ecological concerns. The catalytic systems described herein can be used in applications including synthesis of ester-containing compounds.

One aspect of the technology is a catalytic system that comprises a first catalyst comprising bismuth, aluminum, titanium, zirconium, or combinations thereof, and a second catalyst comprising titanium, zirconium, or combinations thereof.

In some embodiments, the catalytic system comprises from 3 to 96 parts by weight of the first catalyst and from 4 to 97 parts by weight of the second catalyst. In some embodiments, the catalytic system comprises from 5 to 90 parts by weight of the first catalyst and 10 to 95 parts by weight of the second catalyst, from 15 to 80 parts by weight of the first catalyst and 20 to 85 parts by weight of the second catalyst, from 20 to 70 parts by weight of the first catalyst and 30 to 80 parts by weight of the second catalyst, from 25 to 60 parts by weight of the first catalyst and 40 to 75 parts by weight of the second catalyst, from 30 to 50 parts by weight of the first catalyst and 50 to 70 parts by weight of the second catalyst, or from 35 to 40 parts by weight of the first catalyst and 60 to 65 parts by weight of the second catalyst.

In some embodiments, the catalytic system comprises a molar ratio of first catalyst to second catalyst from 1:9 to 4:1. In some embodiments, the catalytic system comprises a molar ratio of first catalyst to second catalyst from 1:9 to 2:1, from 1:9 to 1:1, from 1:9 to 1:2, or from 1:9 to 1:4.

In some embodiments, the catalytic system further comprises water. The water within the catalytic system may be a byproduct of a reaction (e.g., an esterification reaction), a dissolved gas, a solvent, intentionally provided, or any combination thereof. In some embodiments, the catalytic system comprises between 1:1 and 1000:1 equivalents of water to the first catalyst or the second catalyst. In some embodiments, the catalytic system comprises between 1:1 and 900:1, between 1:1 and 800:1, between 1:1 and 700:1, between 1:1 and 600:1, between 1:1 and 500:1, between 1:1 and 400:1, between 1:1 and 300:1, between 1:1 and 200:1, or between 1:1 and 100:1 equivalents of water to the first catalyst or the second catalyst.

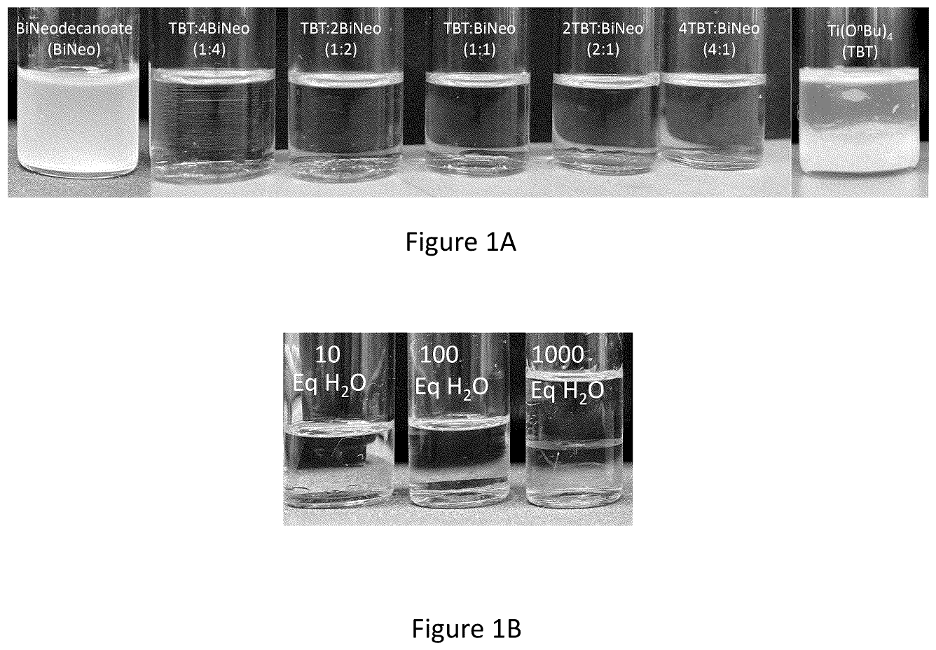

In some embodiments, the catalytic system has less agglomeration than an equivalent metal content of the second catalyst absent the first catalyst. As used herein, the term “agglomeration” refers to a particle formation process in which smaller particles are combined. Agglomeration of catalysts is indicated by formation of clouding or precipitates (e.g. see clouding formation the leftmost or rightmost beaker in FIG. 1A). The amount of agglomeration can be determined qualitatively or quantitatively by visual inspection and/or appropriate analytical methods including, but are not limited to, ultraviolet-visible spectroscopy. The Examples provide exemplary methods for determining agglomeration of catalysts in water.

In some embodiments, the first catalyst comprises bismuth. In some embodiments, the first catalyst comprises Bi(OC(O)R¹)₃, wherein each R¹ is independently an alkyl or aryl. In some embodiments, each R¹ is the same. In other embodiments, two R¹ are the same and a third is different. In yet other embodiments, each R¹ is different. In some embodiments, each R¹ is independently a C₅-C₂₀ alkyl. In some embodiments, one or more of the R¹ is a C₅-C₁₅ alkyl, C₅-C₁₀ alkyl, or C₆-C₉ alkyl. In some embodiments, each R¹ is neododecyl. In some embodiments, the first catalyst comprises bismuth neodecanoate (Bi(C₁₀H₁₉O₂)₃; BiNeo).

In some embodiments, the first catalyst comprises aluminum. In some embodiments, the first catalyst comprises Al(OC(O)R⁴)₃, wherein R⁴ is independently an alkyl or aryl. In some embodiments, each R⁴ is the same. In other embodiments, two R⁴ are the same and a third is different. In yet other embodiments, each R⁴ is different. In some embodiments, each R⁴ is independently a C₁-C₂₀ alkyl. In some embodiments, one or more of the R⁴ is a C₅-C₂₀ alkyl, C₇-C₁₅ alkyl, or C₁₀-C₁₈ alkyl.

In some embodiments, the first, the second, or both the first and second catalyst comprises titanium.

In some embodiments, the first or second catalyst comprises Ti(OC(O)R⁵)₄, wherein each R⁵ is an alkyl or aryl. In some embodiments, each R⁵ is the same. In other embodiments, two R⁵ are the same and the third and fourth are different. In yet other embodiments, three R⁵ are the same and a fourth is different. In yet other embodiments, each R⁵ is different. In some embodiments, each R⁵ is independently a C₃-C₂₀ alkyl. In some embodiments, one or more of the R⁵ is a C₃-C₂₀ alkyl, C₃-C₁₅ alkyl, C₃-C₁₀ alkyl, or C₃-C₆ alkyl. In some embodiments, R⁵ is n-heptyl. In some embodiments, the first or second catalyst comprises titanium octanoate (Ti(C₈H₁₈O₂)₄; TiOct).

In some embodiments, the second catalyst comprises Ti(OR²)₄, wherein each R² is independently an alkyl or aryl. In some embodiments, each R² is the same. In other embodiments, two R² are the same and the third and fourth are different. In yet other embodiments, three R² are the same and a fourth are different. In yet other embodiments, each R² is different. In some embodiments, each R² is independently a C₃-C₂₀ alkyl. In some embodiments, one or more of the R² is a C₃-C₂₀ alkyl, C₃-C₁₅ alkyl, C₃-C₁₀ alkyl, or C₃-C₆ alkyl. In some embodiments, R² is n-butyl. In some embodiments, the second catalyst comprises tetrabutyl titanate (titanium butoxide; Ti(OC₄H₉)₄; TBT).

In some embodiments, the first, the second, or both the first and second catalyst comprises zirconium.

In some embodiments, the first or second catalyst comprises Zr(OC(O)R³)₄, wherein R³ is independently an alkyl or aryl. In some embodiments, each R³ is the same. In other embodiments, two R³ are the same and the third and fourth are different. In yet other embodiments, three R³ are the same and a fourth are different. In yet other embodiments, each R³ is different. In some embodiments, each R³ is independently a C₁-C₂₀ alkyl. In some embodiments, one or more of the R³ is a C₄-C₂₀ alkyl, C₄-C₁₂ alkyl, or C₁₀-C₁₈ alkyl. In some embodiments, the first or second catalyst comprises zirconium octanoate (Zr(C₈H₁₈O₂)₄; ZrOct).

In some embodiments, the second catalyst comprises Zr(OR⁶)₄, wherein each R⁶ is independently an alkyl or aryl. In some embodiments, each R⁶ is the same. In other embodiments, two R⁶ are the same and the third and fourth are different. In yet other embodiments, three R⁶ are the same and a fourth are different. In yet other embodiments, each R⁶ is different. In some embodiments, each R⁶ is independently a C₃-C₂₀ alkyl. In some embodiments, one or more of the R² is a C₃-C₂₀ alkyl, C₃-C₁₅ alkyl, C₃-C₁₀ alkyl, or C₃-C₆ alkyl. In some embodiments, R⁶ is n-butyl. In some embodiments, the second catalyst comprises zirconium tetrabutoxide (Zr(OC₄H₉)₄; TBZr).

As used herein, the term “alkyl” refers to a straight-chain or branched alkyl radical in all of its isomeric forms, such as a straight or branched group of 1-20, 1-15, or 1-10 carbon atoms, referred to herein as C₁-C₂₀-alkyl, C₁-C₁₅-alkyl, and C₁-C₁₀-alkyl, respectively.

As used herein, the term “aryl” refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic.

In some embodiments, the first catalyst comprises bismuth and the second catalyst comprises titanium. In some embodiments, the first catalyst comprises bismuth neodecanoate and the second catalyst comprises titanium butoxide. In some embodiments, the first catalyst comprises bismuth neodecanoate and the second catalyst comprises titanium octanoate.

In some embodiments, the first catalyst comprises bismuth and the second catalyst comprises zirconium. In some embodiments, the first catalyst is bismuth neodecanoate and the second catalyst is zirconium octanoate. In some embodiments, the first catalyst is bismuth neodecanoate and the second catalyst is zirconium tetrabutoxide.

In some embodiments, the first catalyst comprises titanium and the second catalyst comprises titanium. In some embodiments, the first catalyst is titanium octanoate and the second catalyst is titanium butoxide.

In some embodiments, the first catalyst comprises titanium and the second catalyst comprises zirconium. In some embodiments, the first catalyst is titanium octanoate and the second catalyst is zirconium tetrabutoxide. In some embodiments, the first catalyst is titanium octanoate and the second catalyst is zirconium octanoate.

In some embodiments, the first catalyst comprises zirconium and the second catalyst comprises titanium. In some embodiments, the first catalyst is zirconium octanoate and the second catalyst is titanium butoxide.

In some embodiments, the first catalyst comprises zirconium and the second catalyst comprises zirconium. In some embodiments, the first catalyst is zirconium octanoate and the second catalyst is zirconium tetrabutoxide.

In some embodiments, the catalytic system is substantially free, essentially free and/or completely free of catalytic tin.

In some embodiments, the catalytic system is substantially free of catalytic tin. As used herein, the phrase “substantially free of tin” refers to the catalytic system comprising at least less than or equal to 20 wt %, less than or equal to 15 wt %, less than or equal to 10 wt %, less than or equal to 5 wt %, less than or equal to 3 wt %, or less than or equal to 1 wt % of tin.

In some embodiments, the catalytic system is essentially free of catalytic tin. As used herein, the phrase “essentially free of tin” refers to the catalytic system comprising at least less than or equal to 0.5 wt %, less than or equal to 0.3 wt %, less than or equal to 0.1 wt %, less than or equal to 0.05 wt %, less than or equal to 0.01 wt %, or less than or equal to 0.001 wt % of tin.

In some embodiments, the catalytic system is completely free of catalytic tin. As used herein, the phrase “completely free of tin” refers to the catalytic system comprising 0 wt % of tin or an undetectably low level of tin.

In some embodiments, the catalytic system is non-toxic.

The catalytic system is capable of catalyzing an esterification reaction of one or more ester-forming reactants. An ester-forming reactant refers to a compound that can intermolecularly or intramolecularly react to produce an ester bond. The ester-forming reactant comprises one or more groups (e.g., 1, 2, 3, or more) that can react and form an ester bond. Where the ester-forming reactant comprises more than one ester-forming group, the ester-forming groups may be the same or independently selected.

In some embodiments, the one or more ester-forming reactants comprise a compound having a carboxyl group, a hydroxyl group, an anhydride group, or combinations thereof. In some embodiments, the one or more ester-forming reactants comprise a first compound having at least one carboxyl group and a second compound having at least one hydroxyl group. In some embodiments, the one or more ester-forming reactants comprise a first compound having at least one anhydride group and a second compound having at least one hydroxyl group. In some embodiments, the one or more ester-forming reactants comprise a compound having at least one carboxyl group and at least one hydroxyl group. In some embodiments, the one or more ester-forming reactants comprise a compound having at least one anhydride group and at least one hydroxyl group. In some embodiments, the one or more ester-forming reactants comprise a compound having at least one carboxyl group, at least one anhydride group, and at least one hydroxyl group.

In some embodiments, the one or more ester-forming reactants comprise (i) a polyacid, or an ester, or anhydride thereof and (ii) a polyol. In some embodiments, the one or more ester-forming reactants comprise a polyacid and a polyol. In some embodiments, the one or more ester-forming reactants comprise an anhydride of a polyacid and a polyol. In some embodiments, the one or more ester-forming reactants comprise an ester of a polyacid and a polyol.

The terms “carboxyl” refers to the substituent “—COOH”, its corresponding conjugate base, “—COO⁻” or a salt thereof, e.g. —COONa, etc.

The term “hydroxyl” refers to the substituent of “—OH.”

The term “anhydride” as used herein refers to the group

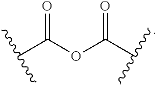 [SMILES: *C(=O)OC(*)=O]

The term “polyacid” refers to a compound that comprises two or more carboxyl functional groups. Examples include, without limitation, dicarboxylic acids such as benzene-1,4-dicarboxylic acid (terephthalic acid).

The term ester of a polyacid refers to a compound that comprises two or more ester functional groups. Examples include, without limitation, diesters such as dimethyl benzene-1,4-dicarboxylate (dimethyl terephthalate).

The term anhydride of a polyacid refers to a compound that comprises an anhydride functional group that can be hydrolized to form a polyacid. Examples include, without limitation, anhydrides such as maleic anhydride.

The term “polyol” refers to a compound that comprises two or more hydroxyl functional groups. Examples include, without limitation, diols such as ethane-1,2-diol.

Another aspect of the technology is to provide a method of preparing the catalytic system as described herein. The method comprises mixing the first catalyst and the second catalyst. In some embodiments, the method further comprises mixing the catalytic system with one or more ester-forming reactants. Suitably, the one or more ester-forming reactants comprise a compound having a carboxyl group, a hydroxyl group, an anhydride group, or combinations thereof. In some embodiments, the one or more ester-forming reactants comprise (i) a polyacid, or an ester or anhydride thereof and (ii) a polyol.

Another aspect of the technology is to provide a method of preparing a compound comprising an ester group. The method comprises reacting one or more ester-forming reactants in the presence of the catalytic system as described herein under conditions sufficient to prepare the compound comprising the ester group. Suitably, the one or more ester-forming reactants comprise a compound having a carboxyl group, a hydroxyl group, an anhydride group, or combinations thereof. In some embodiments, the one or more ester-forming reactants comprise (i) a polyacid, or an ester or anhydride thereof and (ii) a polyol.

In some embodiments, the conditions sufficient to prepare the compound comprising the ester group comprises temperature of 90-350° C. In some embodiments, the conditions sufficient to prepare the compound comprising the ester group comprises temperature of from 95° C. to 340° C., from 100° C. to 330° C., from 105° C. to 320° C., from 110° C. to 310° C., from 115° C. to 300° C., from 120° C. to 290° C., from 125° C. to 280° C., from 130° C. to 270° C., from 135° C. to 260° C., from 140° C. to 250° C., from 145° C. to 220° C., or from 150° C. to 205° C.

In some embodiments, the conditions sufficient to prepare the compound comprising the ester group comprises a contact time of 0.1-30 hours. In some embodiments, the conditions sufficient to prepare the compound comprising the ester group comprises a contact time of from 0.2 hours to 29 hours, from 0.3 hours to 28 hours, from 0.4 hours to 27 hours, from 0.5 hours to 26 hours, from 0.6 hours to 25 hours, from 0.8 hours to 24 hours, or from 1 hour to 24 hours.

In some embodiments, the conditions sufficient to prepare the compound comprising the ester group comprises a pressure of 100-120 kPa. In some embodiments, the conditions sufficient to prepare the compound comprising the ester group comprises a pressure of from 101 kPa to 110 kPa, or from 101 kPa to 105 kPa. In some embodiments, the conditions sufficient to prepare the compound comprising the ester group comprises an atmospheric pressure (i.e. 101.325 kPa).

In some embodiments, the reaction is performed in a solvent comprising xylenes.

In some embodiments, the reaction is performed solventless.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention, and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Examples

### Catalyst Synthesis for TBT:BiNeo Mixtures

Catalysts were synthesized in a glove box with an anhydrous nitrogen atmosphere. A set amount of titanium butoxide (TBT) was mixed with bismuth neodecanoate (BiNeo) to reach the desired molar ratio of titanium to bismuth. The ratios tested included molar ratios of TBT to bismuth neodecanoate of 9:1, 4:1, 2:1, 1:1, 1:2, and 1:4. Full mixing of viscous bismuth neodecanoate and TBT was achieved within 5 minutes as no further changes were seen in the ¹H NMR spectra of mixed catalysts with additional mixing time.

### Catalyst Synthesis for Other Catalytic Mixtures

A set of mixed catalysts were synthesized from common, commercial metal sources. These liquid catalysts were mixed together at various ratios at room temperature. Syntheses were conducted under a dry N₂ atmosphere, although this was found to be unnecessary to form the compounds of interest; synthesis under air indeed showed similar speciation.

Storage of the moisture sensitive Ti precursors was conducted in a dry N₂ glovebox, so synthesis under these conditions was expedient. Some of the mixtures showed a color change from colorless to a shade of yellow to red and a small increase in temperature of 5-15° C. to indicate the exothermic formation of a new compound. Precursor complexes were stirred until the temperature of the mixture had dropped back to room temperature (20-30 min) to ensure a complete reaction.

The resulting viscous oils were stored in amber vials with Teflon caps in a desiccator covered in aluminum foil to prevent excess contact with humidity and possible photoactivity of TiO₂, which could potentially be formed from catalyst hydrolysis. The catalysts were monitored for decomposition into secondary compounds throughout our study, and new catalyst mixtures were regularly synthesized to maintain the primary catalyst species during testing.

### Probe Reaction Experimental

For reactions of phthalic anhydride (PA) with 2-ethylhexanol, 6 mL of 2-ethylhexanol (38.3 mmol) (Sigma-Aldrich, 99%) were added to 1.89 g (12.8 mmol) of phthalic anhydride (Sigma-Aldrich, ≥99.6%) in a 2-neck round bottom flask to give a 3:1 molar ratio of alcohol to anhydride. To the reactants, 0.5 mol %, relative to phthalic anhydride, of undiluted catalyst was added. A Dean-Stark trap was added to one neck of the flask to collect distilled water, and a septum was attached to the second neck. The flask was flushed with N₂ and a low stream of N₂ was added through the reaction via the septum to avoid introduction of ambient moisture. The reaction was then put into an oil bath set to 180° C. and stirred vigorously with a magnetic stirrer bar. Aliquots of 0.1 mL were taken throughout the time-course and conversion was monitored via ¹H NMR with tetramethylsilane as internal standard. The first aliquot was taken at 5 minutes to allow for the conversion of phthalic anhydride to the half-ester intermediate prior to sampling. Further aliquots were taken throughout the reaction progress to monitor conversion trends.

### Stability Testing Experimental

The hydrolytic stability of the catalysts was assessed by adding a set amount of water to a catalyst which had been dissolved in hexane or THF. Catalyst was added to 2 mL of hexane solvent to reach a 1.1 M solution. To the diluted catalyst solution 20 μL of water was added to introduce 10 equivalents of water relative to metal content. The solutions were capped and shaken, and any clouding or precipitation was observed (FIG. 1A).

For stability testing in THF, 2-mL solutions that were 0.1 M in catalyst dissolved in THE with varying amounts of water were prepared. Catalyst was weighed into an empty vial and 2 mL of THE with varying amounts of dissolved water were added. The cloud point was determined by testing against a range of water content amounts and visually observing immediate clouding upon dissolving catalyst in THF/water solution. For higher equivalents of water addition, the above procedure was followed but the volume of water was changed.

### Results Discussion

As demonstrated in FIG. 1A, TBT/BiNeo catalytic systems with varied molar ratios show improved stability in water. After 100% bismuth neodecanoate or 100% titanium butoxide was mixed with 10 equivalents of water, clouding was observed, indicating deactivation or precipitation of the catalyst. However, after 1:4 TBT/BiNeo, 1:2 TBT/BiNeo, 1:1 TBT/BiNeo, 2:1 TBT/BiNeo, or 4:1 TBT/BiNeo was mixed with 10 equivalents of water, little to no visible catalyst precipitation was observed for the TBT/BiNeo catalytic systems.

As shown in FIG. 1B, a TBT/BiNeo catalytic system containing titanium butoxide to bismuth neodecanoate in 1:1 molar ratio was mixed with 10 equivalents, 100 equivalents, or equivalents of water. Surprisingly, very little visible catalyst precipitation was observed even after mixing 1000 equivalents of water with the catalytic system.

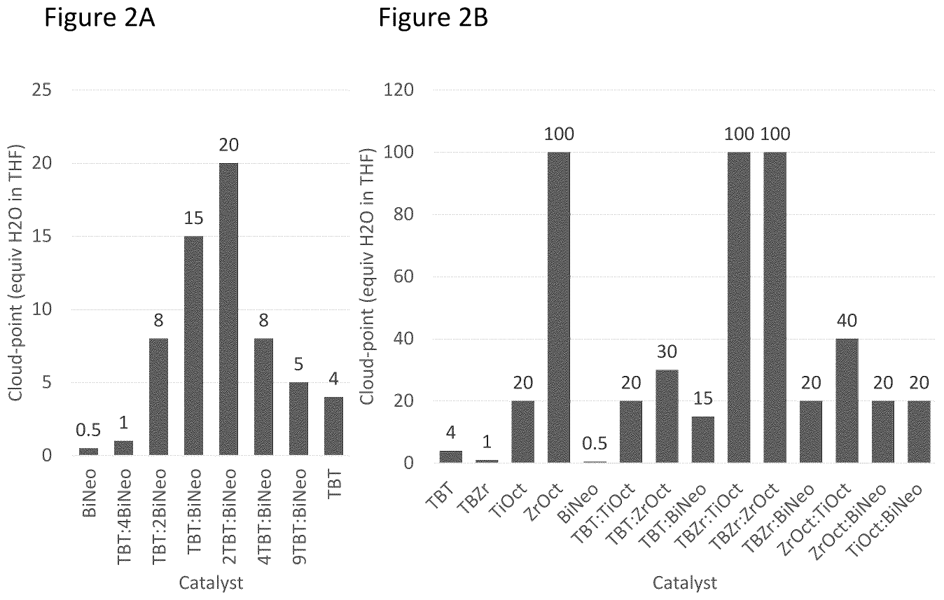

In addition to testing stability of the catalyst in hexane, the stability in THE was also studied. FIG. 2A shows the cloud-point of TBT/BiNeo catalytic systems tested with various equivalents of water at varied molar ratios. At 15 equivalents of water in THF, only 2:1 TBT/BiNeo shows little visible catalyst precipitation. At 20 equivalents of water, clouding is observed for all tested combinations of TBT/BiNeo indicating deactivation of the catalyst.

Stability testing was also done for various catalytic systems as shown in FIG. 2B. TBT, TBZr, and bismuth neodecanoate (BiNeo) have relatively low stability showing precipitation of the catalyst at or below 4 equivalents of water. Titanium octanoate (TiOct), TBT:TiOct, TBZr:BiNeo, ZrOct:TiOct, and TiOct:BiNeo all showed catalyst precipitation at 20 equivalents of water while TBT:ZrOct and ZrOct:TiOct have higher stability. Notably, zirconium octanoate (ZrOct), TBZr:TiOct, and TBZr:ZrOct have even higher stability in THE with water showing no signs of catalyst precipitation at 40 equivalents of water, a cloud-point is instead observed for them when 100 equivalents of water is added. See FIG. 2B and Table 1.

TABLE 1
Water robustness of exemplary catalytic systems.

| | Cloud Point (equiv | | Cloud Point (equiv |
|---|---|---|---|
| Catalyst | H2O in THF) | Catalyst | H2O in THF) |
| BiNeo | 0.5 | BiNeo | 0.5 |
| TBT:4BiNeo | 1 | TBT:TiOct | 20 |
| TBT:2BiNeo | 8 | TBT:ZrOct | 30 |
| TBT:BiNeo | 15 | TBT:BiNeo | 15 |
| 2TBT:BiNeo | 20 | TBZr:TiOct | 100 |
| 4TBT:BiNeo | 8 | TBZr:ZrOct | 100 |
| 9TBT:BiNeo | 5 | TBZr:BiNeo | 20 |
| TBT | 4 | ZrOct:TiOct | 40 |
| TBZr | 1 | ZrOct:BiNeo | 20 |
| TiOct | 20 | TiOct:BiNeo | 20 |
| ZrOct | 100 | | |

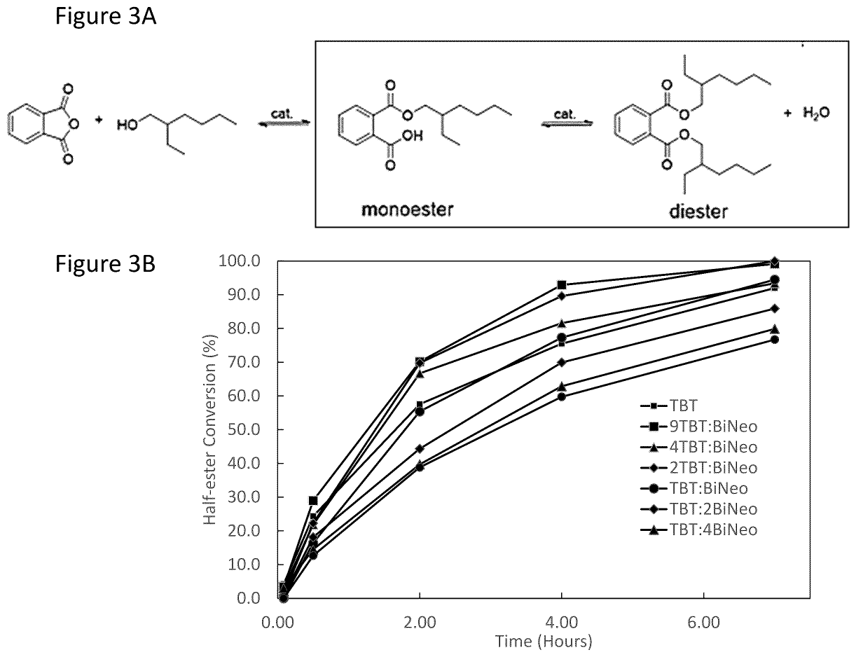

The TBT/BiNeo catalytic system was then subjected to the probe reaction to test the effectiveness of the catalytic system in esterification reactions. See FIG. 3A. As shown in FIG. 3B and Table 2, the TBT/BiNeo catalytic systems having varied TBT and BiNeo molar ratios show variability in conversion. Comparison at hour 7 shows that all of the TBT/BiNeo catalytic systems have greater conversion than bismuth neodecanoate.

TABLE 2
Comparison of TBT/BiNeo catalytic systems with at varied
concentration and the calculated standard error.

| Time | | Std | | Std | | Std | | Std |
|---|---|---|---|---|---|---|---|---|
| (hours) | TBT | Err | 9TBT:BiNeo | Err | 4TBT:BiNeo | Err | 2TBT:BiNeo | Err |
| 0.08 | 4.2 | 1.8 | 3.4 | 0.6 | 3 | 0.6 | 1.1 | 0.7 |
| 0.5 | 24.3 | 7 | 29 | 3.5 | 21.8 | 8.4 | 22.3 | 2.8 |
| 2 | 57.5 | 14.2 | 70.1 | 2.8 | 66.7 | 11 | 69.7 | 3.8 |
| 4 | 75.5 | 10.3 | 92.9 | 4.6 | 81.6 | 0.2 | 89.6 | 0.7 |
| 7 | 92 | 3.9 | 99.1 | 1.2 | 93.4 | 3.6 | 100 | 0 |

| | | Std | | Std | | Std | | Std |
|---|---|---|---|---|---|---|---|---|
| | TBT:BiNeo | Err | TBT:2BiNeo | Err | TBT:4BiNeo | Err | BiNeo | Err |
| 0.08 | 0 | 0 | 0.8 | 1.2 | 3 | 2.1 | 0 | |
| 0.5 | 16.4 | 1.6 | 18.1 | 2 | 14.5 | 2.3 | 12.7 | |
| 2 | 55.4 | 8 | 44.3 | 3.8 | 39.7 | 6 | 38.8 | |
| 4 | 77.3 | 6.9 | 69.9 | 5 | 62.8 | 3.4 | 59.7 | |
| 7 | 94.5 | 2.6 | 85.9 | 9.7 | 79.9 | 3.7 | 76.7 | |

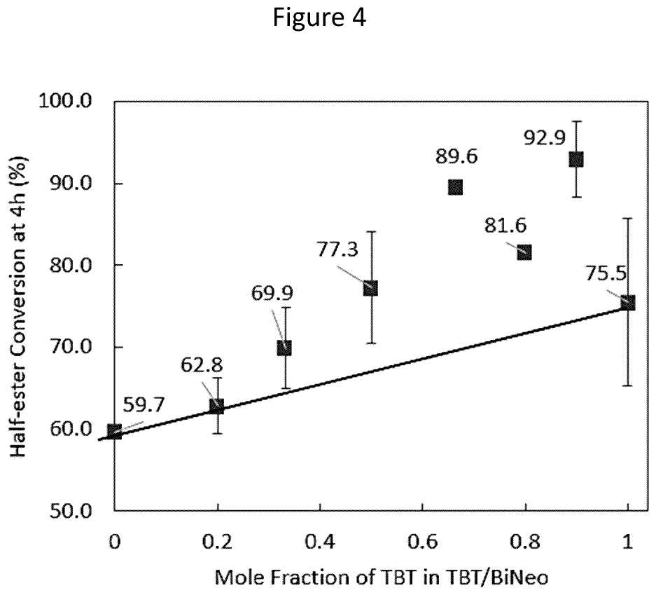

FIG. 4 shows a plot of the percent conversion to the ester product at 4-hour time point using different catalytic systems containing varied mole fractions of titanium. The trendline shows the linear relationship between 0.0 and 1.0 mole fraction of Ti and data points demonstrate that the catalytic system demonstrates superior conversion than would be expected from an admixture. See also Table 3.

TABLE 3
Half-ester conversion at various mole fractions of titanium.

| Mol fraction Ti | Half-ester Conversion [4 h] (%) | StErr |
|---|---|---|
| 1 | 75.5 | 10.2 |
| 0.9 | 92.9 | 4.6 |
| 0.8 | 81.6 | 0.2 |
| 0.67 | 89.6 | 0.7 |
| 0.5 | 77.3 | 6.8 |
| 0.33 | 69.9 | 5.0 |
| 0.2 | 62.8 | 3.4 |
| 0 | 59.7 | — |

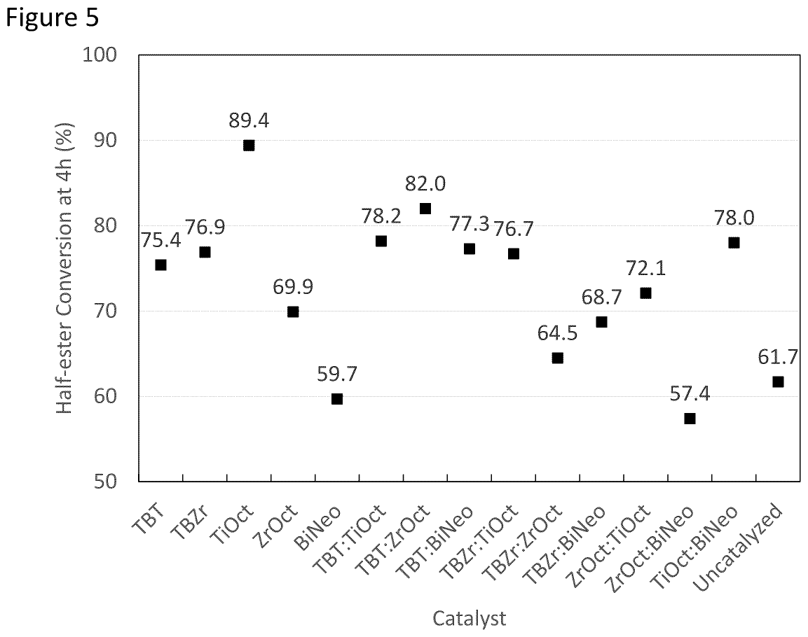

FIG. 5 shows a plot of the percent conversion to the ester product at 4-hour time point using different catalytic systems containing varied catalysts. This plot shows that simple catalytic systems like titanium octanoate can have high conversion at 4 hours (89.40%), while some of the mixed catalytic systems have lower conversions. Table 4 compares the half-ester conversion at 4 hours for all of the catalysts studied. 2TBT:BiNeo and 9TBT:BiNeo have high conversions after 4 hours similar to that of titanium octanoate.

TABLE 4
Half-ester conversion at 4 hours for various
catalytic systems.

| | Half-ester | | Half-ester |
|---|---|---|---|
| | Conversion | | Conversion |
| | at | | at |
| Catalyst | 4 h (%) | Catalyst | 4 h (%) |
| BiNeo | 59.7 | BiNeo | 59.7 |
| TBT:4BiNeo | 62.8 | TBT:TiOct | 78.2 |
| TBT:2BiNeo | 69.9 | TBT:ZrOct | 82.0 |
| TBT:BiNeo | 77.3 | TBT:BiNeo | 77.3 |
| 2TBT:BiNeo | 89.6 | TBZr:TiOct | 76.7 |
| 4TBT:BiNeo | 81.6 | TBZr:ZrOct | 64.5 |
| 9TBT:BiNeo | 92.9 | TBZr:BiNeo | 68.7 |
| TBT | 75.4 | ZrOct:TiOct | 72.1 |
| TBZr | 76.9 | ZrOct:BiNeo | 57.4 |
| TiOct | 89.4 | TiOct: BiNeo | 78.0 |
| ZrOct | 69.9 | Uncatalyzed | 61.7 |

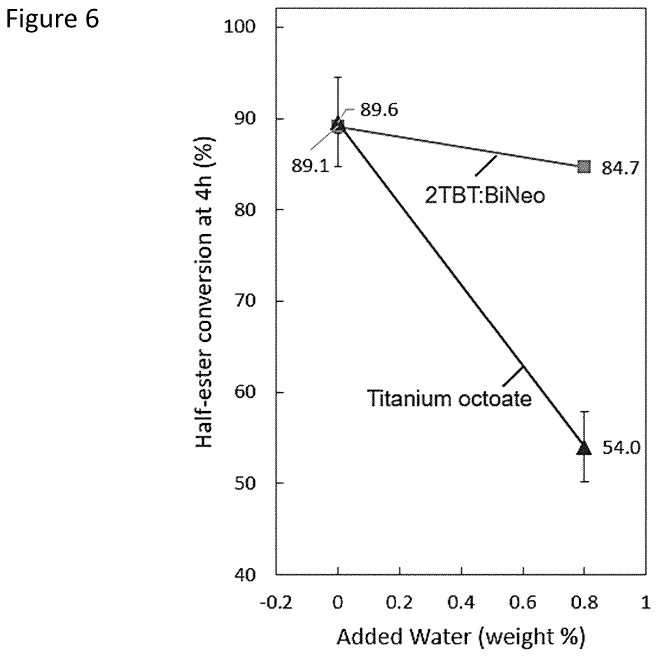

For the 2TBT:BiNeo and titanium octanoate catalytic systems, a reaction with phthalic anhydride started by mixing 1.89 g (12.8 mmol) of phthalic anhydride (PA) with 6 mL of 2-ethylhexanol (38.3 mmol) to give a 1:3 ratio. To the reactants, 0.5 mol %, relative to phthalic anhydride, of catalyst was added prior to heating. Water was added at 0.8 wt % H₂O immediately before starting a reaction. The reaction was then put into an oil bath set to 180° C. and stirred at 300 rpm with a magnetic stirrer bar. The reaction mixture was shown to reach 180° C. roughly 3 minutes after entering the oil bath. Aliquots were taken through the septum and quantified via ¹H NMR with an internal standard. FIG. 6 shows the half-ester conversion at 4 hours with 0.8 weight % of water added compared to half-ester conversion at 4 hours with no water added. While the conversion of the 2TBT:BiNeo catalyst system maintains a high conversion of 84.7%, the conversion with titanium octanoate decreases to 54.0% at 4 hours.

1. A catalytic system comprising a first catalyst comprising bismuth, aluminum, zirconium, titanium, or combinations thereof, and a second catalyst comprising titanium, zirconium, or combinations thereof,
wherein the first catalyst is different from the second catalyst.

2. The catalytic system of claim 1, wherein the catalytic system comprises from 3 to 96 parts by weight of the first catalyst and from 4 to 97 parts by weight of the second catalyst.

3. The catalytic system of claim 1, wherein the catalytic system comprises a molar ratio of first catalyst to second catalyst from 1:9 to 4:1.

4. The catalytic system of claim 1, wherein the catalytic system comprises—
Bi(OC(O)R1)3, wherein each R1 is independently an alkyl or aryl;
Ti(OR2)4, wherein each R2 is independently an alkyl or aryl;
Zr(OC(O)R3)4, wherein each R3 is independently an alkyl or aryl;
Al(OC(O)R4)3, wherein each R4 is independently an alkyl or aryl;
Ti(OC(O)R5)4, wherein each R5 is independently an alkyl or aryl;
Zr(OR6)4, wherein each R6 is independently an alkyl or aryl; or
or any combination thereof.

5. The catalytic system of claim 4, wherein the first catalyst comprises Bi(OC(O)R1)3 and the second catalyst comprises Ti(OR2)4 or Ti(OC(O)R5)4; wherein the first catalyst comprises Bi(OC(O)R1)3 and the second catalyst comprises Zr(OC(O)R3)4 or Zr(OR6)4; wherein the first catalyst comprises Ti(OC(O)R5)4 and the second catalyst comprises Ti(OR2)4; wherein the first catalyst comprises Ti(OC(O)R5)4 and the second catalyst comprises Zr(OC(O)R3)4 or Zr(OR6)4; wherein the first catalyst comprises Zr(OC(O)R3)4 and the second catalyst comprises Ti(OR2)4 or Ti(OC(O)R5)4; or wherein the first catalyst comprises Zr(OC(O)R3)4 and the second catalyst comprises Zr(OR6)4.

6. The catalytic system of claim 4, wherein each R1 is independently a C5-C20 alkyl, each R2 is independently a C3-C20 alkyl, each R3 is independently a C1-C20 alkyl, each R4 is independently a C1-C20 alkyl, each R5 is independently a C3-C20 alkyl, and each R6 is independently a C3-C20 alkyl.

7. The catalytic system of claim 1, wherein the first catalyst comprises bismuth and the second catalyst comprises titanium, wherein the first catalyst comprises bismuth and the second catalyst comprises zirconium, wherein the first catalyst comprises titanium and the second catalyst comprises titanium, wherein the first catalyst comprises titanium and the second catalyst comprises zirconium, wherein the first catalyst comprises zirconium and the second catalyst comprises titanium, or wherein the first catalyst comprises zirconium and the second catalyst comprises zirconium.

8. The catalytic system of claim 1, wherein the first catalyst comprises BiNeo, TiOct, or ZrOct or wherein the second catalyst comprises TBT, TiOct, TBZr, or ZrOct.

9. The catalytic system of claim 8, wherein the first catalyst comprises BiNeo and the second catalyst comprises TBT, wherein the first catalyst comprises BiNeo and the second catalyst comprises TiOct, wherein the first catalyst comprises BiNeo and the second catalyst comprises TBZr, wherein the first catalyst comprises BiNeo and the second catalyst comprises ZrOct, wherein the first catalyst comprises TiOct and the second catalyst comprises TBT, wherein the first catalyst comprises TiOct and the second catalyst comprises ZrOct, wherein the first catalyst comprises TiOct and the second catalyst comprises TBZr, wherein the first catalyst comprises ZrOct and the second catalyst comprises TBT, or wherein the first catalyst comprises ZrOct and the second catalyst comprises TBZr.

10. The catalytic system of claim 1 further comprising water.

11. The catalytic system of claim 10, wherein the catalytic system comprises between 1:1 and 1000:1 equivalents of water to the first catalyst or the second catalyst.

12. The catalytic system of claim 10, wherein the catalytic system has less agglomeration than an equivalent metal content of the second catalyst absent the first catalyst.

13. The catalytic system of claim 1, wherein the catalytic system is substantially free, essentially free and/or completely free of catalytic tin.

14. The catalytic system of claim 1, wherein the catalytic system is capable of catalyzing an esterification reaction of one or more ester-forming reactants.

15. The catalytic system of claim 1 further comprising one or more ester-forming reactants, wherein the one or more ester-forming reactants comprise a compound having a carboxyl group, a hydroxyl group, an anhydride group, or combinations thereof.

16. The catalytic system of claim 14, wherein the one or more ester-forming reactants comprise (i) a polyacid, or an ester or anhydride thereof and (ii) a polyol.

17. A method of preparing the catalytic system according to claim 1 comprising mixing the first catalyst and the second catalyst.

18. The method of claim 17 further comprising mixing the catalytic system with one or more ester-forming reactants, wherein the one or more ester-forming reactants comprise a compound having a carboxyl group, a hydroxyl group, an anhydride group, or combinations thereof.

19. The method of claim 18, wherein the one or more ester-forming reactants comprise (i) a polyacid, or an ester or anhydride thereof and (ii) a polyol.

20. A method of preparing a compound comprising an ester group, the method comprising reacting one or more ester-forming reactants in the presence of the catalytic system according to claim 1 under conditions sufficient to prepare the compound comprising the ester group, wherein the one or more ester-forming reactants comprise a compound having a carboxyl group, a hydroxyl group, an anhydride group, or combinations thereof.

21.-28. (canceled)
